(19) United States
(12) Patent Application Publication
(10) Pub. No.: US 2008/0172037 A1
(43) Pub. Date: Jul. 17, 2008

(54) CATHETER WITH ADJUSTABLE COLUMN STABILITY AND METHODS FOR ITS USE

(75) Inventors: Alex Huang, Menlo Park, CA (US); Rupesh Desai, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Percutaneous Systems, Inc., Mountain View, CA (US)

(21) Appl. No.: 11/555,643

(22) Filed: Nov. 1, 2006

(51) Int. Cl. A61M 25/00

(52) U.S. Cl. 604/526

(57) ABSTRACT

The catheter includes an inner column and an outer elastic member. The inner column is bendable wherein the column strength and flexibility of the inner column are adjusted by axially tensioning the outer elastic member. The inner column comprises a helical spring, slotted or slit tube, or individual links that, when axially compressed by advancement forces or by tensioning an elastic outer sheath, assume a straight or other pre-determined geometry. The column strength and flexibility of the catheter may be adjusted during introduction of the catheter into a body lumen as necessitated by the conditions encountered, as well as made highly flexible when left in position.

### BACKGROUND OF THE INVENTION

1. Field of the Invention

The present invention relates generally to medical apparatus and methods. More particularly, the present invention relates to catheter constructions which provide for controllable rigidity and column strength over the length of the catheter.

Catheters are long tubular devices used for providing access to internal body locations in a wide variety of medical procedures. Catheters will generally include at least one lumen or passageway over their length, and may include additional lumens for particular purposes. Some catheters include interventional or diagnostic components for achieving particular purposes, such as balloons for luminal dilation, optical components for imaging, energy-directing elements for therapy, and the like.

Catheters may be used in a variety of body locations and may be introduced through both solid tissue and luminal passages, such as the vasculature, the ureter, the urethra, nasal passages, the trachea, the cervix, the uterus, the fallopian tubes, and other natural and created body passages. As catheters will be guided through such body passages, it is generally desirable that the catheter bodies be flexible both to accommodate tortuosity and to reduce trauma.

It will be appreciated, however, that medical catheters must also possess sufficient column strength or rigidity to permit them to be advanced through the body passageway. Catheters will typically be advanced by manually pushing their exposed external lengths so that the advancement force is transmitted axially along the length of the catheter to advance the distal end through the body via a sometimes tortuous path. In order to transmit this force without kinking or buckling, the body or shaft of the catheter must possess sufficient column strength to resist the compressive forces of advancement. Additionally, many catheters must also possess a rotational or torsional stiffness sufficient to allow the distal end to be rotated by turning the proximal end. Other desirable and sometimes necessary performance characteristics include hoop strength, fluid carrying capacity, material characteristics, and the like.

Many catheter designs attempt to balance flexibility and column strength by providing proximal portions which are more rigid and distal portions which are more flexible. This is particularly true in coronary and cerebral catheters where the distal regions must enter highly tortuous vasculature while the proximal regions pass through much less tortuous anatomy and therefore may be much less flexible. Micro-catheters used in the cerebral vasculature often have multiple zones of flexibility, where the distal region is highly flexible, an intermediate region has intermediate flexibility, and the proximal portion is more rigid to enhance column strength. The relative amount of flexibility and column strength may be adjusted, zone by zone, by selecting catheter materials having different hardnesses (durometers), providing catheter bodies laminated from different materials, providing different regions of reinforcement selected from coils, braids, and/or fibers, and the like.

While the use of catheters having regions of different flexibility is useful in many instances, it does not address all needs that may be encountered. For example, urinary drainage catheters introduced through the male urethra into the bladder encounter sphincters, strictures, and differing tortuosities which, in some patients, can require a catheter with relatively high axial force transmission for passage. Typical urinary drainage catheters are constructed of elastomeric tubes that rely on increased cross section or more rigid materials to provide adequate handling and efficient force transmission. While many patients might not require such rigid, and therefore uncomfortable drainage catheters, the catheters must be designed for such “worst case” scenarios. Moreover, the need to employ relative rigid catheters often results in a catheter having a large wall thickness, thus reducing the size of the available drainage lumen in that catheter.

In addition to variations in tortuosity between patients, catheters being introduced through body lumens and other passages will frequently encounter localized strictures, obstructions and the like. Each of these may require a relatively high column strength to transmit the higher force necessary for crossing, where such column strength is not required during the remainder of the catheter advancement. Again, present catheters are typically designed with sufficient column strength to address the most demanding portions of their introduction, resulting in excessively rigid and traumatic catheter being advanced during the other portions of the introduction. This compromised flexibility required for the most difficult placements, is particularly troublesome when the catheter is left in place for an extended period, and high flexibility is important for patient comfort.

For these reasons, it would be desirable to provide improved catheters and methods for their use, where the catheters are adaptable to different conditions of luminal introduction. In particular, it would be desirable to provide catheters which have adjustable column strength and flexibility over different region(s) or over their entire lengths. It would be further desirable if such column strength and flexibility could be adjusted by the physician or other user during the performance of a procedure so that differing introduction conditions could be addressed without having to remove the catheter from the patient. Finally, it would be advantageous to have a catheter having sufficient stiffness to traverse a difficult insertion path, but be adjustable to become more flexible, once in position. At least some of these objectives will be met by the inventions described below.

2. Description of the Background Art

Catheters having structures with varying flexibilities and column strengths are described in U.S. Pat. Nos. 4,350,169; 4,464,176; 4,659,328; 4,739,768; 4,861,337; 4,930,521; 5,704,926; 5,919,164; 6,319,244; 7,001,369; 7,001,420; 7,025,758; and published applications US2001/021840 and US2004/034383. Variable stiffness and adjustable guidewires are described in U.S. Pat. Nos. 5,697,380; 5,957,903; 6,113,557; 6,146,339; and 6,183,420. Medical devices having shape lock tubes are described in U.S. Pat. No. 6,790,173. A steerable support catheter having a slotted tube element is described in U.S. Pat. No. 6,746,422.

Catheters and related apparatus having everting sheaths for facilitating catheter introduction are described in copending, commonly owned application Ser. Nos. 10/794,317; 10/794,337; 10/886,886; 10/951,922; 11/233,886; 11/256,562; 11/346,600; 11/367,084; 11/436,256; and 60/821,002, the full disclosures of which are fully incorporated herein by reference.

### BRIEF SUMMARY OF THE INVENTION

The present invention provides medical catheters having adjustable column strength and flexibility over at least a portion or a region thereof. The medical catheters may be of a type used in any one of a wide variety of medical procedures including vascular procedures, urological procedures, gynecological procedures, pulmonary procedures, gastroenterological procedures, endotracheal procedures, and the like. The medical catheters will include a catheter body having a proximal end, a distal end, and at least one lumen therethrough for providing access from a location external to a patient to a location internal to the patient. The catheters will be intended for advancement through a body lumen, typically a natural body lumen such as a blood vessel, a ureter, a urethra, a cervix, a uterus, a fallopian tube, a nasal passage, a trachea, an esophagus, an intestine, a lung passageway, and the like. In other instances, the catheters could be intended for introduction through a created passageway, such as an incision, puncture, or the like made through solid tissue for the purpose of biopsy, therapy, or the like. In most instances, the catheter will have a proximal hub which provides for manipulation of the catheter during its introduction as well as for control of the column strength and flexibility while the catheter is resident in the body lumen. The hub will also usually provide for access to the catheter lumen(s).

As used herein, the phrase “column strength” refers to the ability of the catheter to resist bending, buckling kinking, axial compression and collapse when subjected to a compressive force over its length or any portion of its length. Such compressive forces will typically be encountered as the catheter is being pushed from its proximal end to advance it through the body lumen. The compressive force will result from friction, the presence of tortuosity, strictures, occlusions, sphincters and other natural luminal constrictions, and the like As used herein the term “flexibility” generally means the opposite of column strength and is defined as the inverse of stiffness or the ability to be easily bent. That is, when a lateral force is applied to the catheter, the amount of lateral displacement will vary directly with the degree of flexibility. Thus, a highly flexible catheter will bend easily as it is advanced through tortuous body lumens or encounters strictures or obstructions within the body lumen. Conversely, a catheter having a high column strength will not readily bend, buckle, kink, compress or collapse when advanced through a tortuous body lumen or when encountering a stricture or other obstruction.

In a first aspect of the present invention, a catheter comprises an inner column and an elastic member. The inner column is typically made of a relatively incompressible material and has a distal end, a proximal end, and a lumen therebetween. At least a portion of the column is adapted to stiffen or rigidify in response to axial tensioning in order to increase that portion's column strength. The elastic member is disposed coaxially over the inner column and has a distal end and a proximal end. The distal end of the elastic member is coupled to the column, typically at some point at or near its distal end, and the proximal end of the elastic member is adapted to be axially tensioned, typically by manually pulling or tensioning the member in a proximal direction relative to the column. Thus, applying axial tension to the elastic member will apply compression to the inner column.

The inner column may be constructed in a variety of ways. Most commonly, the inner column will be constructed to straighten and/or to stiffen in response to axial tensioning. In such instances, the inner column of the catheter will still bend when encountering obstructions or luminal tortuosity which applies a lateral bending force to the catheter, but the force required will increase with the amount of axial tensioning In other instances, the inner column will be constructed so that it will assume a predefined curve or other geometry in response to axial tensioning of the elastic member. Such designs may be desirable, for example, to provide steerable catheter configurations, where rotation of the catheter body can selectively direct the distal end of the catheter into side branches, conform to curved tracts such as a male urethra or other luminal locations as the catheter is advanced. For both the straightened and curved catheter designs, increasing the axial tensioning force applied by the elastic member will increase the stiffness or rigidity of the straight or curved geometry, thus making the catheter more pushable.

In the specific embodiments described below, the inner column will comprise a plurality of adjacent links which are able to pivot relative to each other in response to a lateral bending force. Typically, the adjacent links will engage each other or “nest” in response to the axial tensioning force applied by the elastic member. The geometry of the adjacent links will determine the geometry of the column assumed under tension, typically straight or curved as discussed above. The column links may be provided by a variety of specific column constructions, including helical coils (optionally having varying cross-sectional shapes), slit tubes, slotted tubes, hellically slit tubes, nested rings, and the like, as described more fully below. The contact surfaces between links can also affect flexibility as the tension is adjusted, e.g. flat surfaces brought into opposition under tension will resist bending/provide more column strength than will curved contact surfaces.

The elastic member may be any structure which is coaxially disposed about the inner column and which provides for application of axial tension on the column as a proximal portion of the elastic member is pulled proximally relative to the column. Typically, the elastic member will comprise a tubular member having a continuous surface which forms a fluid tight seal or constraint around the catheter structure. Alternatively, the elastic member could be a lattice, a braid, or other open or foraminous structure which could be penetrable or allow the passage of body fluids through the wall of the catheter.

Most commonly, the tubular member will comprise a continuous tubular body composed at least partly of an elastomeric polymer. In other instances, however, the tubular member could comprise a non-elastic tubular body having one or more elastic components or reinforcements to provide for elasticity along at least a portion of the length of the body.

The tubular member may extend substantially over the entire length of the inner column or in other instances may be attached at a location proximal to the distal end of the column. In still other instances, the tubular member may extend distally beyond the distal end of the inner column, in which case the tubular member may provide a distal tip or attached region of the catheter of different deflection characteristics or geometry, for example, providing an atraumatic curved tip, a region for diagnostic or interventional tools or an anchoring balloon.

A hub(s) will usually be attached to the proximal end(s) of the inner column and/or the elastic member. Usually, the hub includes a mechanism for axially tensioning the elastic member relative to the inner column. For example, the tensioning mechanism may comprise a slide member, a screw mechanism, ratchet, snap or a variety of other mechanical devices for allowing manual or automatic adjustment of the tensioning. The tensioning mechanism could alternatively be attached directly to the elastic member, for example as a ring or a collar, to permit proximal translation.

In other embodiments, the catheter may further comprise a distal tip which is attached to the distal end of the inner catheter and/or elastic member. The catheter may also further comprise a sheath positioned in the lumen of the inner catheter, where the sheath is able to evert from the distal end of the catheter as the catheter is advanced through a body lumen. In still further embodiments, the catheter could comprise a liner in the lumen of the inner catheter, where the liner could provide for isolation of a lumen from the surrounding body environment.

The ability of the elastic member to “elastically” tension the inner column is particularly advantageous since it provides for adjustment of the column strength and flexibility of the catheter over a wide range from very flexible when the elastic member is very loose or slack over the inner column to a very high stiffness or column strength when the elastic member is pulled to a maximum tension. Preferably, the elastic member will be fabricated from natural or synthetic rubber elastomers such as silicone, latex, urethane, fluorocarbon elastomers or thermoplastic elastomers with low compression set and and elongation in the range from 100% to 1400%, usually 200% to 1000%, and preferably from 300% to 800%.

It should be appreciated that even when the inner column of the catheter is under axial compression applied by the elastic member, the catheter will retain a degree of flexibility since the elastic member will be able to yield laterally when the catheter encounters vessel tortuosity or an obstruction. The degree and nature of flexibility in yielding will, of course, be first a function of the geometry of the intersection of the links, and further dependent on the degree of axial force applied by the elastic member and the degree of elasticity in the elastic member itself, and both these latter quantities may, of course, be adjusted by the user by pulling or axially translating the proximal end of the elastic member relative to the inner column.

In a second aspect of the present invention, a method for advancing a catheter through a body lumen comprises pushing the catheter from a proximal portion outside of the body lumen to advance a distal portion of the catheter through the lumen. While the distal portion of the catheter is within the body lumen, the stiffness of at least a portion of the catheter may be adjusted by axially tensioning an elastic member located coaxially over an inner column. The body lumen may be selected from a group consisting of ureters, urethras, blood vessels, an intestine, nasal passages, a lung passage, the esophagus, the trachea, the cervix, the uterus, the fallopian tubes, and the like. The catheter may be positioned over a guidewire as it is pushed through the body lumen, but will often be advanced without the use of the guidewire.

Pushing the catheter typically comprises manually advancing a hub attached to a proximal portion of the catheter. Alternatively, pushing may comprise robotically advancing a hub attached to a proximal portion of the catheter. In such instances, the robot and its attached control system could automatically control the tension applied by the elastic member to the inner column in order to control the catheter stiffness in response to the conditions encountered.

Adjusting the catheter stiffness will usually comprise stiffening the catheter when more column strength is needed to pass through an obstruction or stricture in the body lumen. Usually, stiffening will comprise straightening at least a portion of the catheter. In other instances, however, stiffening may comprise inducing a curve into at least a portion of the catheter, where the curve may often be used for steering to target regions within the body lumen.

Alternatively, adjusting could comprise reducing the stiffness of the catheter when the catheter is being pushed through a tortuous region of the body lumen to allow conformance of the catheter body to the lumen or in creating a highly flexible and comfortable device once placed into its proper position. This unique ability to reduce stiffness, and thereby increase comfort, is particularly important where a catheter requiring significant pushability to place in position, will be left in that position, within a patient, for an extended period of time.

In the preferred embodiments, the catheter will comprise the catheter structures described above. That is, the inner column will typically comprise a plurality of adjacent links which pivot relative to each other in response to laterally applied bending forces resulting from the catheter engaging a luminal wall, obstruction, or stricture if the catheter is pushed. Axial tensioning of the elastic member will cause the adjacent links to nest or otherwise to resist pivoting and increase the column strength of the inner column. Even in the absence of tension in the elastic member, axial compression of the catheter resulting from the pushing of catheter during advancement may generate forces that will cause the elements of the inner column to resist pivoting and nest in their most axially compact (straight or curved) geometry. The amount of axial compression will vary while the catheter lumen is pushed through the body lumen depending on the particular conditions encountered. In the case where the fully nested form of the inner links is a straight column, axial compression from advancement, (in addition to any tension introduced by the elastic member), will increase column strength of the catheter for improved pushability and efficient transmission of forces through the catheter.

It may be disadvantageous for portions of the catheter, particularly the distal end of the catheter, to become more stiff when compressed. In such cases, the inner nesting elements may terminated proximal to the tip, inner nesting elements may be spaced apart and bonded to the elastic sleeve to prevent solid stacking, or the geometry of the inner elements may be changed to preserve lateral pivoting under compression. Further optionally, the methods may still further comprise everting a sheath over a distal portion of the catheter as it is pushed through the body lumen. The methods may further optionally comprise performing an interventional procedure through the catheter after the catheter has been placed at the target lumen. Still further optionally, the methods may further comprise performing a diagnostic procedure through the catheter after the catheter has been placed at a target location. As in the above, the methods may further comprise reducing the amount of axial tensioning in order to provide a softer, more comfortable catheter for long-term placement within a patient.

### BRIEF DESCRIPTION OF THE DRAWINGS

FIG. 1 illustrates a first catheter construction comprising an inner column and an outer elastic member constructed in accordance with the principles of the present invention.

FIG. 2 illustrates the catheter structure of FIG. 1 responding to a laterally applied bending force.

FIG. 3 illustrates a second embodiment of the catheter structure of the present invention, where the catheter structure will assume a curved geometry when the elastic member applies a tensioning force to the inner column.

FIG. 4 illustrates the catheter structure of FIG. 3 responding to a laterally applied force.

FIG. 5 illustrates a distal tip which may be attached to the catheter structures of the present invention.

FIG. 6 illustrates a lumen liner which may be incorporated in the catheter structures of the present invention.

FIGS. 7-11 illustrate different inner column structures including a helical coil (FIG. 7), a slit tube (FIGS. 8-9), a slotted tube (FIG. 10), and nested rings (FIG. 11).

FIG. 12 illustrates the catheter structure where the outer elastic member is attached at the distal end of the inner column.

FIG. 13 illustrates a catheter structure where the outer elastic member is attached at a location proximal to the distal end of the inner column.

FIG. 14 illustrates a catheter structure where the outer elastic member extends distally beyond the distal end of the inner column.

FIG. 15 illustrates a screw-type mechanism for tensioning the outer elastic member relative to the inner column by advancing/compressing the inner column

FIG. 16 illustrates a slide-type mechanism for tensioning the outer elastic member relative to the inner column by advancing/compressing the inner column

FIG. 17 illustrates a catheter structure in accordance with the principles of the present invention further having an everting sheath for facilitating introduction to a body lumen.

### DETAILED DESCRIPTION OF THE INVENTION

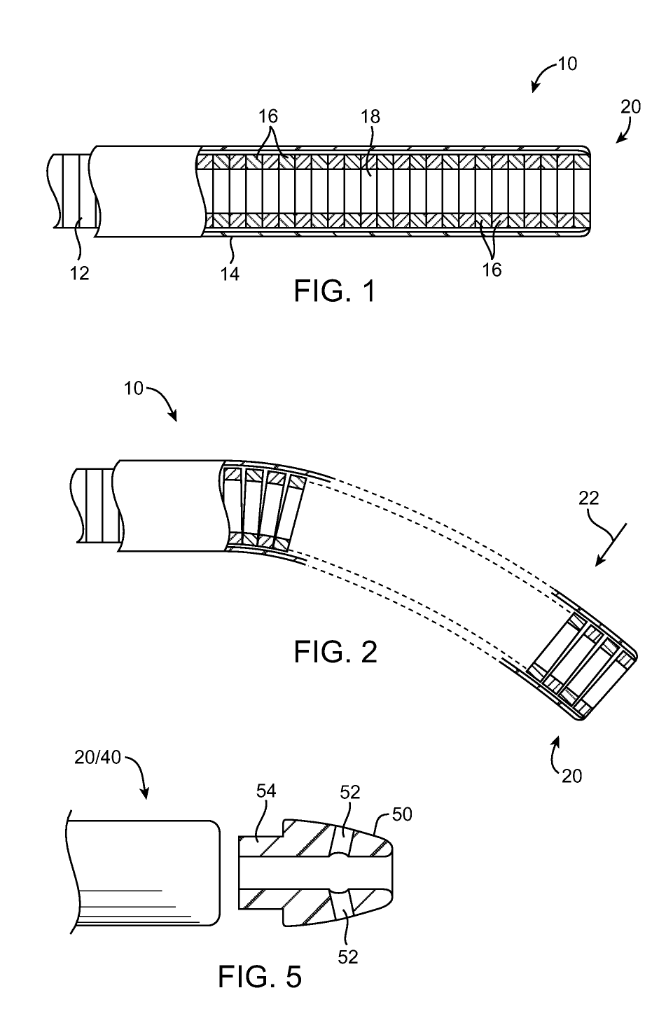

Referring to FIGS. 1 and 2, catheter structure 10 constructed in accordance with the principles of the present invention includes an inner column 12 and an outer elastic member 14. The inner column 12 comprises a plurality of adjacent or “stacked” links 16 which assume a generally straight configuration when axially tensioned by the elastic member 14, as shown in FIG. 1. The links 16 may be unattached and held together only by the axial tensioning of the outer member 14. In other instances, the links 16 may comprise a continuous structure, such as a helical coil, a slotted tube, a slit tube, and the like, as described in more detail hereinafter. Noncontinuous structures, such as the individual links 16 illustrated in FIG. 1, may optionally be held together by tethers, coupling elements, or other components which would provide some degree of structural integrity in the absence of the elastic member 14. Elastic member 14, however, will be the principal element intended to provide the variable or elastic tensioning force to the links in the present invention.

The links 16 shown in FIG. 1 will be located at a distal end 20 of the catheter structure 10. The links could extend for the entire length of the catheter, or the more proximal portions of the catheter could be formed from conventional structures, such as continuous polymeric tubes, hypo-tubes, or the like. In some instances, it may be disadvantageous for portions of the catheter, particularly the distal end of the catheter, to become stiff when compressed, such as when a narrowing or torturosity is encountered. The selectively flexible portions of the catheter could also be located at regions other than the distal tip of the catheter, although it is generally preferred that increased flexibility be provided at or near the distal end of the catheter.

The catheter structure 10 will be able to bend in response to a lateral bending force as indicated by arrow 22 in FIG. 2. The amount of force needed to bend the catheter and/or the degree of bending in response to a given lateral force will depend on the amount of axial tensioning which is being provided by the elastic member 14. When the elastic member 14 is being tensioned with a relatively greater force, the inner column 12 will have a relatively greater column strength (stiffness or rigidity) and a lesser degree of flexibility. Conversely, when the axial tensioning force is reduced, the inner column will have a lesser column strength and a greater flexibility. Thus, the physician or other user will be able to adjust the column strength and flexibility of the catheter structure 12 as it is being advanced through a body lumen depending on the conditions which are encountered. When the body lumen is generally free from obstructions and strictures, the column strength can be reduced so that the catheter, particularly the distal region of the catheter remains flexible and less likely to cause trauma to the patient. Conversely, if a structure or obstruction is encountered in the body lumen, the column strength (stiffness or rigidity) of the catheter 10 may be increased to facilitate pushing and advancing the catheter past the obstruction or stricture. This ability to adjust the column strength of the catheter allows the physician or other user to use the minimum column strength or stiffness necessary to continue to advance the catheter, thus enhancing the comfort of the patient and reducing the risk of traumatic injury.

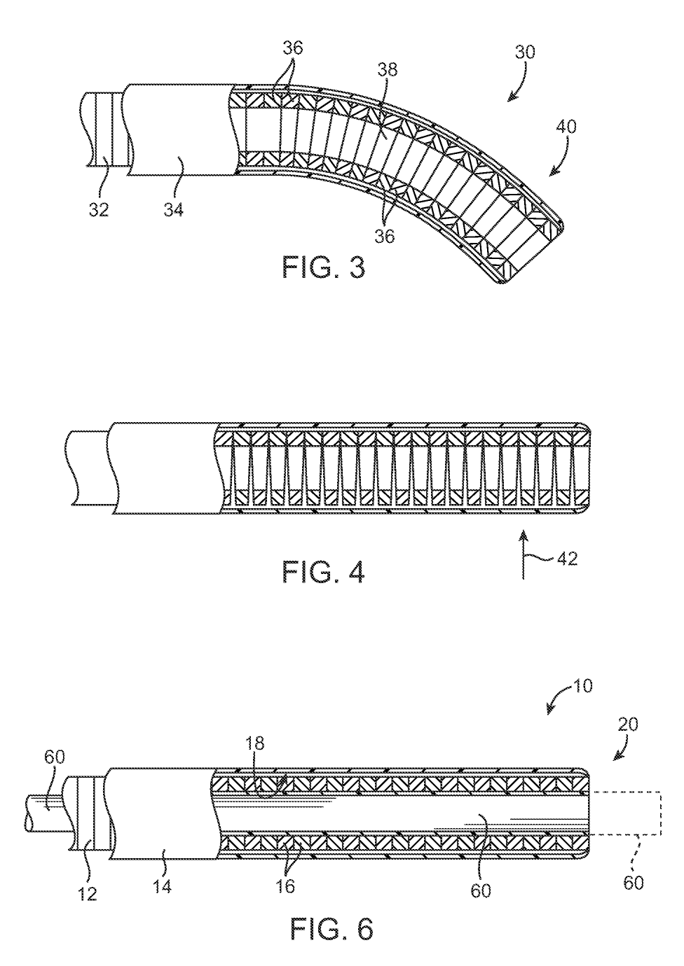

Referring now to FIGS. 3 and 4, an alternative catheter structure 30 comprises an inner column 32 and outer elastic member 34. In contrast to the catheter structure 10 of FIGS. 1 and 2, individual links 36 of the catheter structure 30 have a wedge-shaped profile so that the distal region 40 of the catheter will have a curved or arcuate profile, as shown in FIG. 3, when proximal tension is applied to the outer elastic member 34. Such arcuate shape or profile may be useful in a variety of circumstances, such as when it is desired to steer or direct the distal end 40 through a branching or sharply curved luminal structure by rotating the catheter body about its axis. The curved structure shown in FIG. 3 may be generally straightened by a lateral force as indicated by arrow 42 in FIG. 4. The amount of lateral force required to straighten the catheter (or to bend the catheter in any other direction) will depend on the amount of axial tensioning which is being provided by proximal pulling of the elastic member 34, and the amount of compression introduced by resistance to advancement forces.

The catheter structures 10 and 30 may have additional components, including a distal tip 50 as shown in FIG. 5. The distal tip 50 may have a tapered or bullet-shaped profile for assisting in the atraumatic advancement of the catheter through a body lumen. The tip 50 may further include infusion or diffusion ports 52 to help distribute fluids, or alternatively to aspirate or drain body fluids. The distal tip 52 may include a proximal collar 54 which is received in the distal end 20/40 of the catheter structures.

The catheter structures of the present invention may also include a liner 60 disposed within the lumen 18/38 of the catheter structures 10 and 30. As shown in FIG. 6, the liner 60 is disposed in the straightened lumen 18 of catheter structure 10, optionally terminating at the distal end of the lumen (as shown in full line) or extending distally beyond the distal end (as shown in broken line) with other components in catheter structure 10 remaining the same as described above. The inner columns of the catheter structures of the present invention may take a wide variety of specific forms.

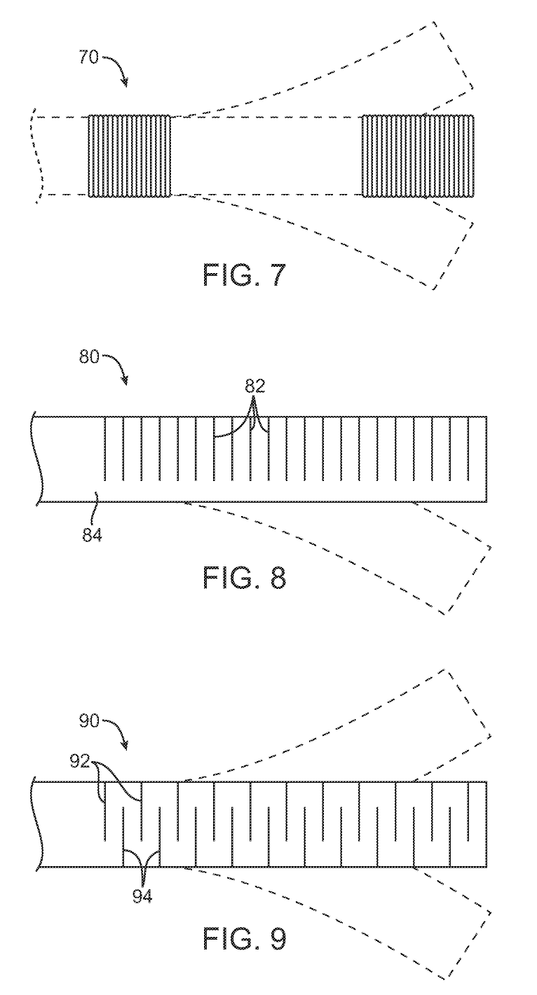

As described above, the columns may comprise a plurality of discrete links or other elements which are held together in a column structure by the outer elastic member and/or tethers or other retaining structures. In many cases, however, it would be preferable to provide an inner column which is defined by a continuous structure which is inherently flexible but which returns to a straight or other pre-defined geometry upon the application of axial tension. As shown in FIG. 7, the inner column may comprise a helical coil having a plurality of adjacent turns, where the turns act as links which close together or nest when subjected to an axial tensioning force, such as that provided by the elastic member of the present invention. The turns of the coil, however, can separate to allow the helical coil to deflect in any direction, as shown in broken line in FIG. 7. The degree of bending and the force required to induce the bending will depend, of course, on the degree of axial tension being provided by the elastic member when the helical coils 70 are incorporated into the catheter structures.

An alternative inner column in the form of a slotted tube 80 is illustrated in FIG. 8. The tube 80 may comprise flexible, but relatively incompressible material, such as Nitinol, stainless steel, high density polyethylene or the like. A plurality of slots 82 may be formed along one side of the tube by laser cutting or other conventional techniques. When the slots are arranged along one side of the tube, and those slots are sufficiently deep, a “spine” 84 remains which allows the tube to be bent in one direction, as shown in broken line in FIG. 8, when a lateral force is applied. With certain materials such as Nitinol, the slit tube 80 will return to the straight configuration shown in full line in FIG. 8, when the lateral force is removed.

As shown in FIG. 9, an alternative slit tube 90 may comprise individual slits 92 along one side and other slits 94 in the opposite direction along the other side. Inclusion of such staggered slits 92 and 94 allow an inner column formed from tube 90 to flex in two opposed directions, as shown in broken line in FIG. 9. It will be appreciated that lateral slits could be formed in still further orientations, allowing additional bending directions in the resulting tubular column.

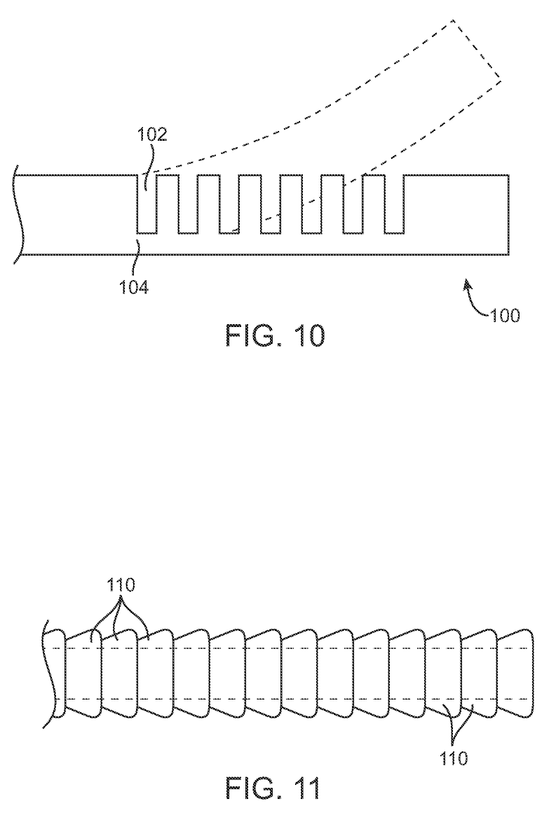

As shown in FIG. 10, tubular column 100, formed from Nitinol, stainless steel, or the like, could be provided with a series of axial slots 102 extending from a “spine” 104 which allows the tube to bend either in the direction shown in broken line, where the slots are axially collapsed, or in the opposite direction where the slots are allowed to open further.

As shown in FIG. 11, the inner column of the present invention could also be formed from a plurality of nested rings 110, where the individual rings are able to articulate relative to each other to allow a high degree of flexibility when the rings are not subjected to a high amount of axial tensioning. The rings will be configured so that, when axial tensioning is applied to the resulting column, the adjacent rings will assume a pre-defined configuration, shown as a stiffened linear or straight configuration in FIG. 11. Curved or other configurations would also be possible, depending on how the individual rings are configured. The amount of resistance to a lateral force could, in this case, be tailored by the amount of nesting allowed between adjacent rings. One could have a region of relatively high flexibility between rings with a round cross-section. At the same time and degree of axial tension, one could have high stiffness in another region, where a tapered male section of one ring fits into a tapered female section of the adjacent element.

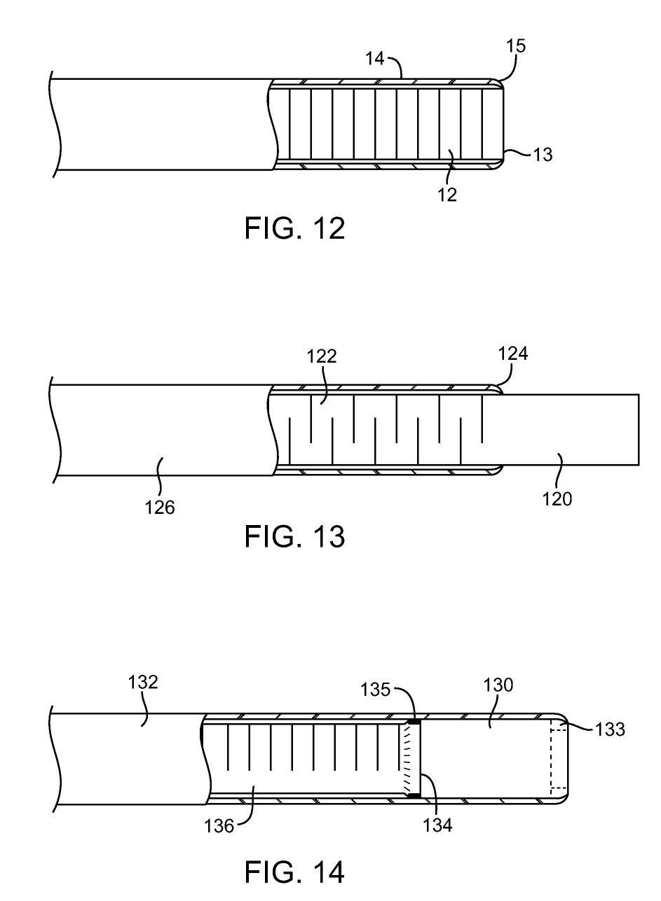

Referring now to FIGS. 12-14, the inner column and elastic member will be joined or otherwise coupled to each other at some point along their respective lengths. The column and elastic member will be coupled in order to transmit an axial tensioning force from the elastic member to the inner column when the elastic member is pulled or translated in a proximal direction relative to the inner column. As the elastic member is pulled proximally (or the inner column advanced relative to the elastic member), the amount of axial tensioning force applied to the inner column will increase from a very low initial level to increasingly greater levels as the relative axial displacement increases.

Typically, as shown in FIG. 12, a distal end 15 of the outer elastic member 14 will be attached to the distal end 13 of the inner column 12. This is the same attachment configuration shown in FIGS. 1-4. In some instances, however, it may be desirable to extend a distal end 120 of an inner column 122 beyond the distal end 124 of an outer elastic sleeve 126, as shown in FIG. 13. The extended distal end 120 of the column could provide for an enhanced region of stiffness at the distal end if that were desired. Alternatively, it could provide a platform for attaching an interventional, diagnostic, or other tool (not shown) used in a particular procedure. Still further alternatively, a distal end 130 of an outer elastic tube 132 may be configured to extend distally beyond the distal end 134 of an inner column 136, as shown in FIG. 14. The elastic tube 132 is attached to the inner column at some point proximal from the distal end of the column, e.g. by an adhesive 135 at the circumference of the distal end 134. The distal end 130 of the elastic member 132 may provide for an atraumatic distal tip, or for other desirable characteristics in the resulting catheter structure. In all of these embodiments, proximal tensioning of the outer elastic member will be able to apply axial compression to the portion of the inner column which lies proximally of the attachment point.

In some cases it may be desirable to be able to partially or completely remove the inner column from the catheter. In such embodiments, an abutment stop 133 (broken line, FIG. 14) on the inside of the elastic tube 132 of the catheter could be provided in lieu of a permanent attachment point. The abutment stop 133 may engage the distal end 134 of the inner column 136 or may engage a corresponding shoulder, ferrule or flange (not shown) located at any position along the length of the inner column. Multiple sets of stops and abutments could be used to create differing stiffness profiles depending on rotation and tension.

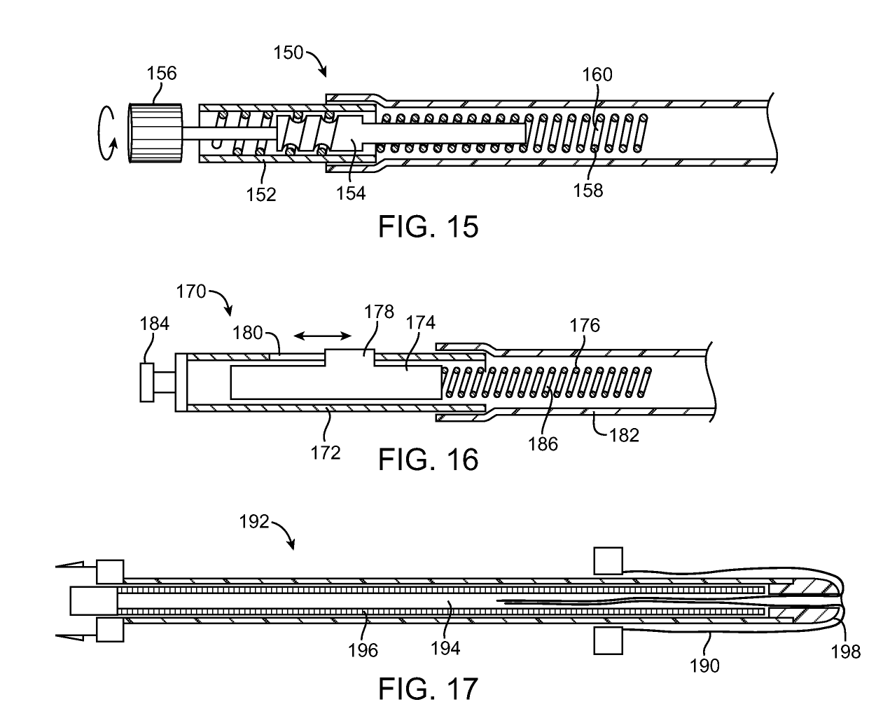

Referring now to FIGS. 15 and 16, the catheter structures of the present invention will usually be provided with proximal hubs or other structures for effecting proximal tensioning of the outer elastic member relative to the inner column. For example, as shown in FIG. 15, a hub structure 150 comprises an internally threaded housing 152 having a rotatable externally threaded element 154 therein. A handle 156 is attached to the threaded element, and the element, in turn, is attached to a proximal end of an inner column 158, shown as a helical coil. Preferably, the threaded element 154 will be able to rotate relative to the inner column 158, and passage will be provided through the structure so that access to lumen 160 within the inner column 112 may be attained. Axial tensioning of the column 158 is adjusted by rotating the handle 156 in a clockwise or counterclockwise direction.

An alternative hub structure 170 is shown in FIG. 16, where a tubular housing 172 slidably receives a slide element 174. The slide element is attached to the proximal end of an inner column 176, again shown as a helical coil. A thumb element 178 passes through an axially slotted opening 180 in the housing 172 to permit the user to manually advance and retract the inner column 176 relative to the outer elastic member 182. A luer or other fitting 184 is provided on the housing 172 to allow for access to the inner lumen 186 of the inner column 176.

Referring now to FIG. 17, in some instances it may be desirable to provide an evertable sheath 190 to facilitate introduction of a catheter structure 192 into a body lumen. The evertable sheath 190 is initially held within lumen 194 of inner column 196 of the catheter structure 192. As the catheter structure 192 is advanced, the sheath is pulled from the lumen, everting around the distal tip 198 to facilitate introduction of the catheter structure into the body lumen. The use of such sheaths is well described in the various copending applications of Assignee of the present application, which are referred to and incorporated by reference above.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A catheter comprising:
an inner column having a distal end, a proximal end, and a lumen therebetween, wherein at least a portion of the column stiffens in response to axial tensioning; and
an elastic member disposed coaxially over the inner column, said elastic member having a distal end coupled to the inner column and a proximal end adapted to be axially tensioned, wherein applying axial tension to the proximal end of the elastic member will stiffen the inner column.

2. A catheter as in claim 1, wherein the inner column straightens in response to axial tensioning.

3. A catheter as in claim 1, wherein the inner column assumes a predefined curve in response to axial tensioning.

4. A catheter as in claim 1, wherein the inner column comprises a plurality of adjacent links which can pivot relative to each other in response to a laterally applied bending force.

5. A catheter as in claim 4, wherein the links nest with each other in response to an axially applied compressive force.

6. A catheter as in claim 5, wherein the links arrange along a straight line when axially compressed and resist bending in response to laterally applied bending forces.

7. A catheter as in claim 5, wherein the links arrange along a curved line when axially compressed and resist bending in response to laterally applied bending forces .

8. A catheter as in claim 4, wherein the inner column comprises a helical coil.

9. A catheter as in claim 4, wherein the inner column comprises a tube having lateral slits.

10. A catheter as in claim 4, wherein the inner column comprises a tube having lateral slots.

11. A catheter as in claim 4, wherein the inner column comprises a tube having a helical slit.

12. A catheter as in claim 1, wherein the elastic member comprises a tubular member.

13. A catheter as in claim 12, wherein the tubular member comprises a tubular body composed at least partly of an elastomeric polymer.

14. A catheter as in claim 12, wherein the tubular member comprises a non-elastic tubular body having elastic reinforcement.

15. A catheter as in claim 12, wherein the tubular member extends over substantially the entire length of the inner column.

16. A catheter as in claim 12, wherein the tubular member extends beyond the distal end of the inner column.

17. A catheter as in claim 16, where the tubular member is attached to the distal end of the inner column, proximal to the distal end of the tubular member.

18. A catheter as in claim 16, wherein the tubular member is attached to the inner column at a location proximal to the distal end of the inner column.

19. A catheter as in claim 12, wherein the inner column is removably abutted against a stop in a lumen of the tubular elastic member.

20. A catheter as in claim 1, further comprising a hub attached to the inner column.

21. A catheter as in claim 20, wherein the inner column is removable from the elastic member and engages an abutment on the elastic member.

22. A catheter as in claim 20, wherein the hub includes a mechanism for axial tensioning the elastic member over the inner column.

23. A catheter as in claim 22, wherein the mechanism comprises a slide mechanism.

24. A catheter as in claim 22, wherein the mechanism comprises a screw mechanism.

25. A catheter as in claim 1, further comprising a distal tip attached to the distal end of the inner column and/or of the elastic member.

26. A catheter as in claim 1, further comprising a sheath positioned in the lumen of the inner column to evert from the distal end of the catheter as the catheter is advanced through a body lumen.

27. A catheter as in claim 1, further comprising a liner in the lumen of the inner column.

28. A method for advancing a catheter through a body lumen, said method comprising:
pushing the catheter from a proximal portion outside the body lumen to advance a distal portion through said lumen; and
adjusting the stiffness of at least a portion of the catheter by axial tensioning an elastic member located coaxially over an inner column to selectively stiffen at least a portion of the inner column.

29. A method as in claim 28, wherein the stiffness is adjusted while the catheter is outside the body lumen.

30. A method as in claim 28, wherein the stiffness is adjusted while the catheter is in the body lumen.

31. A method as in claim 28, wherein the body lumen is selected from the group consisting of ureters, urethras, blood vessels, the gastrointestinal tract, nasal passages, pulmonary bronchii, the esophagus, the trachea and lumens created through solid tissue.

32. A method as in claim 28, wherein the catheter is positioned over a guidewire as it is pushed through the body lumen.

33. A method as in claim 28, wherein pushing comprises manually advancing a hub attached to the proximal portion of the catheter.

34. A method as in claim 28, wherein pushing comprises robotically advancing a hub attached to the proximal portion of the catheter.

35. A method as in claim 28, wherein adjusting comprises stiffening the catheter when more column strength is needed to pass an obstruction or stricture in the body lumen.

36. A method as in claim 28, wherein adjusting comprises reducing the stiffness of the catheter when the catheter is being pushed through tortuous regions of the body lumen.

37. A method as in claim 28, wherein adjusting comprises reducing the stiffness of the catheter to increase patient comfort after placement.

38. A method as in claim 28, wherein adjusting comprises compressing the catheter to straighten at least a portion thereof.

39. A method as in claim 28, wherein adjusting comprises compressing the catheter to induce a curve in at least a portion thereof.

40. A method as in claim 28, wherein the inner column comprises a plurality of adjacent links which pivot relative to each other in response to laterally applied bending forces resulting from the catheter engaging a luminal wall, obstruction, or stricture as the catheter is pushed.

41. A method as in claim 40, wherein axial tensioning the elastic member causes the adjacent links to nest to resist pivoting and increase the column strength of the inner column.

42. A method as in claim 41, wherein the amount of axial tensioning is varied at different times while the catheter is pushed through the body lumen.

43. A method as in claim 28, further comprising everting a sheath over the distal portion of the catheter as it is pushed through the body lumen.

44. A method as in claim 28, further comprising performing an interventional procedure through the catheter after the catheter has been placed at a target location.

45. A method as in claim 28, further comprising performing a diagnostic procedure through the catheter after the catheter has been placed at a target location.
